(19) United States
(12) Patent Application Publication
(10) Pub. No.: US 2008/0261276 A1
(43) Pub. Date: Oct. 23, 2008

(54) Micro-Fluidic Device Based Upon Active Matrix Principles

(75) Inventors: Marc Wilhelmus Gijsbert Ponjee, Eindhoven (NL); Henrik Roelof Stapert, Eindhoven (NL); Mark Thomas Johnson, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/067,347

(22) PCT Filed: Sep. 22, 2006
(86) PCT No.: PCT/IB06/53434

§ 371 Date: Mar. 19, 2008

(30) Foreign Application Priority Data
Sep. 23, 2005 (EP) 05108796.3

(51) Int. Cl. C12P 19/34; B01J 19/00; G01N 21/00

(52) U.S. Cl. 435/91.2; 422/68.1; 422/82.12; 73/866

(57) ABSTRACT

A micro-fluidic device (1) including a two-dimensional array of a plurality of components (2) for processing a fluid and/or for sensing properties of the fluid is suggested. Each component (2) is coupled to at least one control terminal (9,10) enabling an active matrix to change the state of each component individually. The components comprise at least one heater element (13). The active matrix includes a two-dimensional array of electronic components (12) realized in thin film technology. The active matrix provides a high versatility of the device. The thin film technology ensures a very cost efficient manufacturing also of large devices.

### FIELD OF THE INVENTION

The present invention is related to a micro-fluidic device including a two-dimensional array of a plurality of components for processing a fluid and/or for sensing properties of the fluid.

### BACKGROUND OF THE INVENTION

Micro-fluidic devices are at the heart of most biochip technologies, being used for both the preparation of fluidic samples and their subsequent analysis. The samples may e.g. be blood based. As will be appreciated by those in the art, the sample solution may comprise any number of things, including, but not limited to, bodily fluids like blood, urine, serum, lymph, saliva, anal and vaginal secretions, perspiration and semen of virtually any organism: Mammalian samples are preferred and human samples are particularly preferred; environmental samples (e.g. air, agricultural, water and soil samples); biological warfare agent samples; research samples (i.e. in the case of nucleic acids, the sample may be the products of an amplification reaction, including both target an signal amplification); purified samples, such as purified genomic DNA, RNA, proteins etc.; unpurified samples and samples containing (parts of) cells, bacteria, virusses, parasites or funghi.

As it is well known in the art, virtually any experimental manipulation may have been done on the sample. In general, the terms “biochip” or “Lab-on-a-Chip” or alike, refer to systems, comprising at least one micro-fluidic component or biosensor, that regulate, transport, mix and store minute quantities of fluids rapidly and reliably to carry out desired physical, chemical and biochemical reactions in larger numbers. These devices offer the possibility of human health assessment, genetic screening and pathogen detection. In addition, these devices have many other applications for manipulation and/or analysis of non-biological samples. Biochip devices are already being used to carry out a sequence of tasks, e.g. cell lyses, material extraction, washing, sample amplification, analysis etc. They are progressively used to carry out several preparation and analysis tasks in parallel, e.g. detection of several bacterial diseases. As such, micro-fluidic devices and biochips already contain a multiplicity of components, the number of which will only increase as the devices become more effective and more versatile.

Many of the components are electrical components used to sense or modify a property of the sample or fluid, such as heating elements, pumping elements, valves etc., and are frequently realized by direct fabrication of thin film electronics on the substrate of the device. Suitable properties that can be sensed or modified include, but are not limited to, temperature; flow rate or velocity; pressure, fluid, sample or analyte presence or absence, concentration, amount, mobility, or distribution; an optical characteristic; a magnetic characteristic; an electrical characteristic; electric field strength, disposition, or polarity.

One problem of this approach is that every electrical component on the device requires control terminals to independently control the component. Consequently, more space is required to connect the components to the control devices than to realize the devices themselves. Ultimately, the number of control terminals will become so large that it will become impractical to arrange all the terminals at the periphery of the device to make electrical contact. One possibility to realize the electrical contact is the use of an electrical contact foil.

In numerous biotechnological applications, such as molecular diagnostics, there is a need for biochemical processing modules, comprising an array of temperature controlled reaction compartments that can be processed in parallel and independently to allow high versatility and high throughput. In many of these applications, the analysis system consists of a (disposable) cartridge (e.g. biochip, lab-on-a-chip, microfluidic device or alike system) comprising a biochemical processing module and a bench-top machine. In many of such biochemical systems the components for temperature control as well as analysis (e.g. light source, CCD camera, etc.) are located in the bench-top machine instead of on the, often disposable, cartridge. A major drawback of this approach is that the bench-top machine can only be used for a particular design or a selective number of cartridge designs. Consequently, the performance of various assays requires nowadays a plurality of bench-top machines.

In order to avoid a large number of control terminals, U.S. Pat. No. 6,852,287 proposes embodiments of a method to control a number N of independently controllable components with smaller number of control terminals. In order to achieve this, both the use of multiplexing techniques or passive matrix techniques is proposed. In particular, the matrix technique is extremely attractive, as this allows for the maximum number of components to be controlled with the minimum number of control terminals. Conceptually, if one specific heater element is activated also a number of other heater elements will be activated unintentionally. As a result, heat will be generated where it is not required, and the heat generated at the intended heater element will be different than required as either some of the applied current has traveled through alternative paths, or the applied voltage is dropped along the rows and columns before reaching the heater element intended to be activated.

It is an object of the invention to provide a micro-fluidic device having an improved performance compared to passive matrix based devices. This object is achieved by a micro-fluidic device, e.g. a biochip, fabricated on a substrate based upon active matrix principles. The device is preferably fabricated from one of the well known large area electronics technologies, such as a-Si, LTPS or organic transistor technologies. The active matrix makes it possible to independently control a larger number of components on the device with a smaller number of control terminals.

It is a further object of the invention to provide an array of temperature controlled reaction compartments that can be processed in parallel and independently, in a cost-effective way on a biochip or alike system, without the need for a large device periphery to locate the I/O pins.

### SUMMARY OF THE INVENTION

The present invention describes a micro-fluidic device including a two-dimensional array of a plurality of components for processing a fluid and/or for sensing properties of the fluid. The components comprise at least one heater element. Each component is coupled to at least one control terminal enabling an active matrix to change the state of each component individually. The active matrix includes a two-dimensional array of electronic components realized in thin film technology. The active matrix provides a high versatility of the device. The thin film technology ensures a very cost efficient manufacturing also of large devices.

In a preferred embodiment, the device comprises at least two, even more preferred a multiplicity of heater elements. Such a device is referred to as a thermal processing array. These heater elements are suitable for heating fluid that may be present in cells or compartments of the microfluidic device.

The thermal processing array can be used to either maintain a constant temperature across the entire compartment area, or alternatively to create a defined time-dependent temperature profile if the reaction compartment is also configured in the form of an array and different portions of the reaction chamber require different temperatures.

In a most preferred embodiment, the thermal processing array comprises a multiplicity of individually addressable and drivable heating elements, and may preferably comprise additional elements such as temperature sensors and fluid-mixing or fluid-pumping elements or a combination thereof.

The inclusion of at least one temperature sensor is highly preferred. Even more preferred, the device comprises a multiplicity of temperature sensors to control a pre-defined temperature profile across an array of components or cells. This embodiment is illustrated in FIG. 4.

Preferably, the components for heating, and the other optional components, are all present on a biochemical processing module, which is preferably located in a biochip, lab-on-a-chip, microfluidic device, or alike system. The micro-fluidic device is preferably a disposable unit, which may be a replaceable part of a larger disposable or non-disposable unit (e.g. lab-on-a-chip, genechips, microfluidic device, or alike system). In addition to the components, the device may optionally comprise cells or cavities that can hold a fluid. Such cells are also referred to as array elements.

In one advantageous embodiment of the invention the electronic components of the active matrix are formed by thin film transistors having gate, source and drain electrodes. In this case the active matrix includes a set of select lines and a set of control lines such that each individual component is controlled by one select line and one control line and the gate electrode of each thin film transistor is connected to a select line.

In another advantageous embodiment of the invention a memory device is provided for storing a control signal supplied to the control terminal.

In an alternative embodiment of the invention the electronic components are formed by thin film diodes, e.g. metal-insulator-metal (MIM) diodes. It is preferred that a MIM diode connects a first electrode of each component to a control line, and a second electrode of each component is connected to a select line.

In another advantageous embodiment of the invention the thin film diodes are PIN or Schottky diodes, wherein a first diode connects a first electrode of each component to a control line, wherein a second diode connects the first electrode of each component to a common rest line and wherein a second electrode of each component is connected to a select line.

In an advantageous development of the invention the first diode is replaced by a pair of diodes connected in parallel and the second diode as well is replaced by a pair of diodes connected in parallel.

In yet another advantageous development the first diode is replaced by a pair of diodes connected in series, and also the second diode is replaced by a pair of diodes connected in series.

### BRIEF DESCRIPTION OF THE DRAWINGS

The invention will be better understood and other particular features and advantages will become apparent on reading the following description appended with drawings. In the drawings:

FIG. 1 a schematic block diagram of a micro-fluidic device according to the invention illustrating the active matrix concept; At least one of the components (2) is a heater element.

FIG. 2 a first embodiment of the micro-fluidic device, the active matrix of which is based on thin film transistors; At least one of the components (2) is a heater element.

FIG. 3 shows a device wherein it is possible to sequentially activate heaters in different lines.

FIG. 4 shows a microfluidic device comprising a multiplicity of temperature sensors.

### DESCRIPTION OF PREFERRED EMBODIMENTS

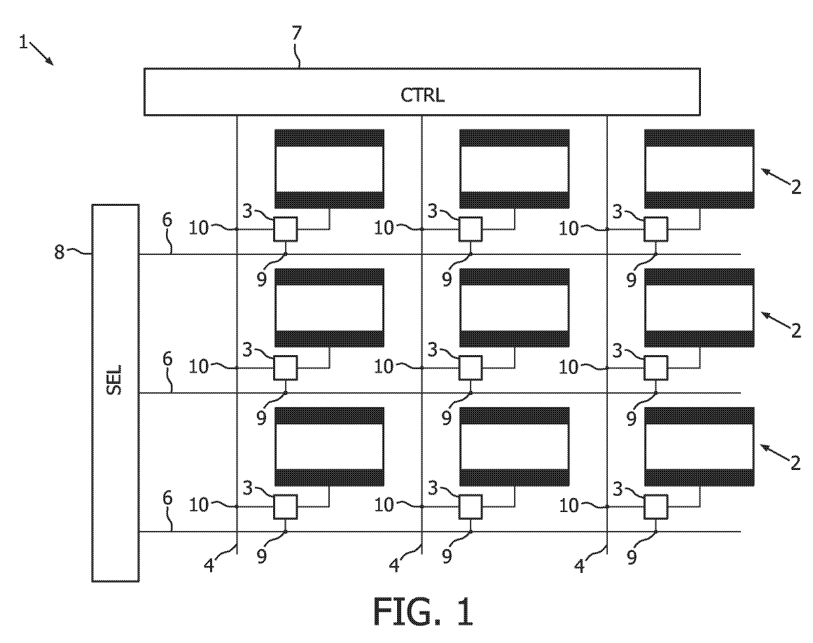

FIG. 1 illustrates the general concept of a micro-fluidic device based on an active matrix. The micro-fluidic device as a whole is designated with the reference number 1. The device comprises a two-dimensional array of components 2. Each component 2 is associated with a switching means 3 arranged to selectively activate the component 2. Each switching means is connected to a control line 4 and a select line 6. The control lines 4 are connected to a common control driver 7. The select lines 6 are connected to a common select driver 8. The control lines 4 in conjunction with the select lines 6 form a two-dimensional array of control terminals 9, 10.

In this way an active matrix is realized to ensure that all components can be driven independently. The component 2 may be any electronic device e.g. a heater element, a pumping element, a valve, a sensing component etc. being driven by either a voltage or a current signal. It is to be understood that the examples for the components 2 are not to be construed in a limiting sense. Activating a component 2 means changing its state e.g. by turning it from on to off, or vice versa or by changing its setting. It is also noted that the individual switching means 3 may comprise a plurality of sub components comprising both active and/or passive electronic components. However, there is no requirement that all sub components are activated together.

The operation of the micro-fluidic device 1 illustrated in FIG. 1 to independently control a single component 2 is as follows:
In the non-addressing state, all select lines 6 are set to a voltage where the switching elements 3 are non-conducting. In this case, no component 2 is activated.
In order to activate a preselected component 2 the select driver 8 applies a select signal to the select line 6 to which the preselected component 2 is coupled. As a consequence all switching means 3 connected to the same select line 6 are switched into a conducting state.
A control signal generated by the control driver 7, e.g. a voltage or a current is applied to the control line where the preselected component 2 is situated. The control signal is set to its desired level and is passed through the switching means 3 to the component 2, causing the component to be activated.
The control signals in all other control lines 4 are held at a level, which will not change the state of the remaining components connected to the same select line 6 as the preselected component 2. In this example, they will remain un-activated.
All other select lines 6 will be held in the non-select state, so that the other components 2 connected to the same control line 4 as the preselected component will not be activated because their associated switching means 3 remain in a non-conducting state.
After the preselected component is set into the desired state, the respective select line 6 is unselected, returning all switching means 3 into a non-conducting state, preventing any further change in the state of the preselected component.

The device will then remain in the non-addressed state until the following control signal requires to change the state of any one of the components 2, at which point the above sequence of operation is repeated.

The two-dimensional array formed by the control lines 4 and the select lines 6 can also be described in terms of rows and columns, where the select lines 6 define the rows and the control lines 4 the columns.

It is also possible to control more than one component 2 in a given row simultaneously by applying a control signal to more than one column in the array during the select period. It is possible to sequentially control components in different rows by activating another row by using the select driver and applying a control signal to one or more columns in the array.

It is also possible to address the micro-fluidic device 1 such that a component 2 is only activated while the control signal is present. However, in a preferred embodiment, it is advantageous to incorporate a memory device into the component whereby the control signal is remembered after the select period is completed. For the memory device a capacitor or a transistor based memory element is suitable. This makes it possible to have a multiplicity of components at any point across the array activated simultaneously. This option is not available in the passive system known in the prior art. Of course, if a memory device is available, a second control signal will explicitly be required to de-activate the component.

Preferably the device comprises cells and channels, most preferred microfluidic channels, that connect one cell to at least one, or more preferred a plurality of, other cells. Optionally a valve is located between the cells. This enables the performance of a reaction with various steps in the device. In such an embodiment, fluids may be moved sequentially from one cell to another or alternatively many cells may be processed in parallel.

In particular, the invention enables accurate, reproducible, reliable and fast thermal cycling during DNA amplification on a biochip, for instance using (multiplexed) PCR or (multiplexed) real-time quantitative PCR (RQ-PCR), such that the temperature of the array elements may be individually and in parallel controlled, without significant additional costs or issues concerning the number of input and output pins. Moreover, with respect to the situation in which the heating elements are located in the bench-top machine, this invention offers a more optimal and more reliable thermal contact between temperature components and fluid.

Therefore in a further aspect the invention relates to use of the device according to the invention in a process wherein temperature is controlled.

In another aspect the invention relates to use of the device according to the invention in a process wherein the temperature is changed according to a pre-defined regime.

Last but not least, this invention allows an advantageous way of performing RQ-PCR on a biochip by combining a cost-effective high performance thermal processing array (e.g. high resolution, individual and parallel temperature control of compartments, high reproducibility, high reliability and high accuracy) on the disposable, with the high performance (e.g. high resolution, high signal-to-noise ratio) of an optical detection setup (e.g. light source, CCD camera, filters) generally used in a bench-top machine for detection of fluorescent signals in molecular diagnostics.

Hence in a further aspect the invention relates to a method of performing the PCR process, preferably RQ-PCR process wherein use is made of the micro-fluidic device as described above.

In another aspect the invention relates to the microfluidic device as described above, in combination with an optical detection set up.

In a further aspect the invention relates to a method of detecting a product using a diagnostic device comprising a micro-fluidic device according to the invention, wherein the detection is based on optical methods.

After having illustrated the general concept and the advantages of a micro-fluidic device 1 in the following description specific embodiments will be explained.

The first embodiment describes an active matrix micro-fluid device based on thin film transistors

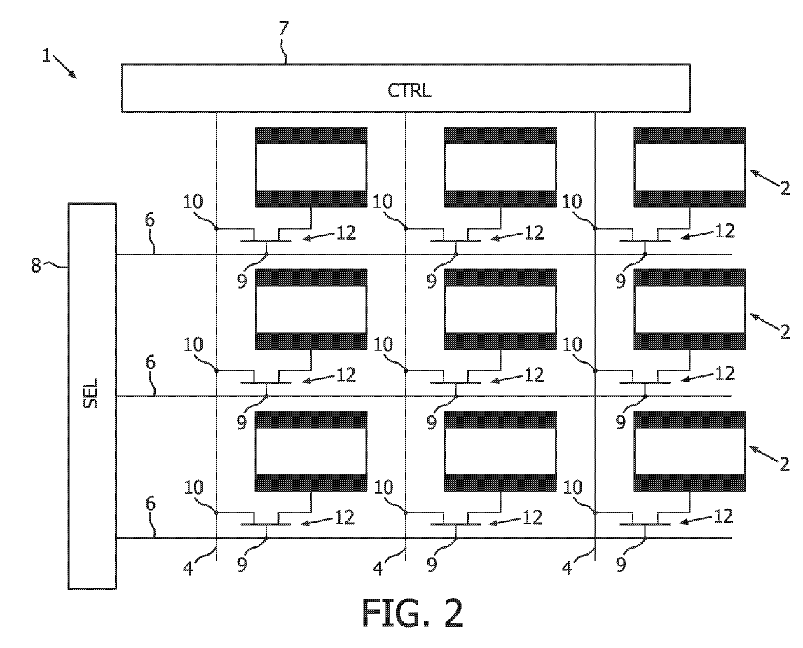

FIG. 2 exhibits an active matrix micro-fluidic device 1 using thin film transistors (TFT) 12 as switching means 3 to ensure that all components, for example the heating elements, can independently be activated. Each component 2 is connected to the matrix of control terminals via a TFT switch 12. At least one of the components is a heater element. TFTs are well known switching elements in thin film large area electronics, and have found extensive use e.g. in flat panel display applications. Industrially, the major manufacturing methods for TFTs are based upon either amorphous-silicon (a-Si) or low temperature polycrystalline silicon (LTPS) technologies. But other technologies such as organic semiconductors or other non-Si based semiconductor technologies, such as CdSe, can be used. The operation of the device illustrated in FIG. 2 to independently control a single component 2 is as follows:
In the non-addressing state, all select lines 6 are set to a voltage where the TFTs are non-conducting. In the case of a-Si, we have typically an n-type TFT and hence a negative voltage has to be applied to the gate of the TFTs. In this case, no component 2 is activated.
In order to activate a preselected component 2 the select driver 8 applies a positive select signal to the select line 6 to which the preselected component 2 is connected. Thus, all TFTs 12 connected to this select line are switched into their conducting state.
A control signal generated by the control driver 7, a voltage or current signal is applied to the column where the preselected component is located. The TFT 12 passes the control signal to the preselected component, which is coupled to the drain of the TFT, for activating the component.
The control signals in all other columns are held at a level that will not change the state of remaining components of the row. In this example, they will remain un-activated.
The select signals of all other rows will be held in the non-select state by applying a negative voltage signal to the gate of the TFTs, so that the other components are connected to the same column via non-conducting TFTs and will not be activated.
After the component is set into the desired state, the TFTs 12 in the row are again set to the non-conducting state, preventing any further change in the state of the component.

The device will then remain in the non-addressed state until the following control signal requires to change the state of any one of the components, at which point the above sequence of operation is repeated.

With a TFT based switch, it is again possible to control more than one component in a given row simultaneously by applying a control signal to more than one column in the array during the select period. It is possible to sequentially control components in different rows by activating another row by using the select driver and by applying a control signal to one or more columns in the array. Furthermore, it is still possible to address the system such that the component is only activated while the control signal is present, or alternatively to incorporate a memory device into the component (e.g. a capacitor element, or a transistor based memory element) whereby the control signal is remembered after the select period is completed.

In another embodiment, the heater elements are provided as a regular array of identical units, whereby the heaters are connected to the driver via the switches (e.g. transistors) of the active matrix. The gates of the transistors are connected to a select driver (for example a standard shift register gate driver as used for an AMLCD), whilst the source is connected to the heater driver, for example a set of voltage or current drivers. Operation is as follows:
To activate a given heater element, the transistors in the line incorporating the required heater are switched into the conducting state (by e.g. applying a positive voltage to the gates from the select driver).
The signal (voltage or current) in the column where the heater is situated is set to its desired value. This signal is passed through the conducting TFT to the heater element, resulting in a local temperature increase.
The driving signal in all other columns is held at a voltage or current, which will not cause heating (this will typically be 0V or 0 A).
After the temperature increase has been realized, the transistors in the line are again set to the non-conducting state, preventing further heater activation.

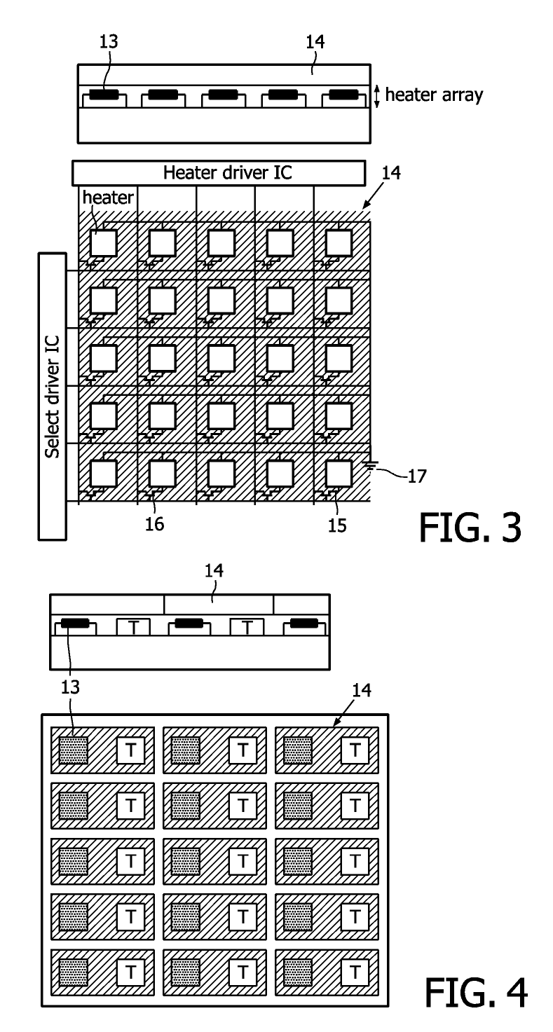

It is also possible to activate more than one heater in a given line simultaneously by applying a signal to more than one column in the array. It is possible to sequentially activate heaters in different lines by activating another line (using the gate driver) and applying a signal to one or more columns in the array. This embodiment is illustrated in FIG. 3. In a first embodiment the biochemical processing module comprises a discrete array of heating elements (13) based on active matrix principles, such that a reaction compartment (14) contains a plurality of heaters. In FIG. 3 the following is shown:

heating element (13)

reaction compartment (14)

transistor switch (16)

heater electrode (15)

common electrode (17)

There are several options for configuring the biochemical processing module depending upon the required heat processing.

Whilst in this embodiment of FIG. 3 a driver is considered which is capable of providing (if required) signals to all columns of the array simultaneously, it is also feasible to consider a more simple driver with a function of a de-multiplexer. In this example (FIG. 5a) only a single output driver is required to generate the heating signals (e.g. a voltage or a current). The function of the de-multiplex circuit is simply to route the heater signal to one of the columns, whereby only the heater is activated in the selected line in that column. It should be understood that a plurality of drivers with a function of a de-multiplexer may be used to drive the entire heater array.

In another embodiment according to the invention, an integrated heater driver is included per heating elements based on active matrix technology.

In yet another embodiment, the device comprises a local driver provided with a memory function. This allows the heating signal to be applied for a longer period of time, enabling better and more accurate control of a given temperature profile.

The inclusion of at least one temperature sensor is highly preferred. Even more preferred, the device comprises a multiplicity of temperature sensors to control a pre-defined temperature profile across an array of components or cells. This embodiment is illustrated in FIG. 4.

In this embodiment (FIG. 4), the biochemical processing module comprises a compartment (14) or a plurality (e.g. array) of compartments (14) and a discrete array of heating elements (13) and the at least one temperature sensor (T). Each heating element is individually drivable, whereby a multiplicity of temperature profiles may be created. The temperature profile can advantageously be measured by a plurality of temperature sensors.

In embodiments, the temperature sensors may be used to prevent a temperature from extending beyond a given range, and may preferably be used to define and control the desired temperature profile.

In another embodiment the device comprises a compartment or plurality (e.g. array) of compartments (cells) and a discrete array of heating elements and at least one mixing or pumping element. Each heating element is individually drivable, whereby a multiplicity of temperature profiles may be created. A uniform temperature profile can advantageously be created by a plurality of mixing or pumping elements.

In a preferred embodiment, the mixing or pumping elements are integrated into the heating element array, for example if this component were to be manufactured using large area thin film electronics technologies, such as low temperature Poly-Si.

To make the device especially suitable for a PCR process, it is advantageous to include a cooling element. In an embodiment, cooling is provided by a (bench-top) machine handling a PCR module, for instance by bringing the PCR module in thermal contact with a cooled mass, peltier element, etc., or by use of convection (e.g. fan). In another embodiment, a cooling element is incorporated in the PCR module, such as a thin-film peltier element, or an array of cooling elements is incorporated.

1. Micro-fluidic device (1) comprising a two-dimensional array of a plurality of components (2) for processing a fluid and/or for sensing properties of the fluid, wherein the components comprise at least one heater element, and wherein each component (2) is coupled to at least one control terminal (9, 10) enabling an active matrix to change the state of each component individually, and wherein the active matrix includes a two-dimensional array of electronic components realized in thin film technology.

2. Micro-fluidic device (1) according to claim 1, wherein the electronic components of the active matrix are formed by thin film transistors having gate, source and drain electrodes.

3. Micro-fluidic device (1) according to claim 2, wherein the active matrix includes a set of select lines (6) and a set of control lines (4) such that each individual component (2) is controlled by one select line (6) and one control line (4) and in that the gate electrode of each thin film transistor is connected to a select line (6).

4. Micro-fluidic device (1) according to claim 1, wherein a memory device is provided for storing a control signal supplied to the control terminal (9, 10).

5. Micro-fluidic device (1) according to claim 1 comprising at least two heater elements (13).

6. Micro-fluidic device (1) according to claim 1, comprising a multiplicity of individually addressable and drivable heater elements (13).

7. Micro-fluidic device (1) according to claim 1, further comprising a temperature sensor.

8. Micro-fluidic device (1) according to claim 1 further comprising a cooling element.

9. Micro-fluidic device (1) according to claim 1, comprising mixing and/or pumping elements.

10. Method of performing a PCR process, preferably RQ-PCR process, wherein use is made of the micro-fluidic device according to claim 1.

11. Use of the device according to claim 1 in a process wherein temperature is controlled.

12. Method of detecting a product using a diagnostic device comprising a micro-fluidic device according to claim 1, wherein the detection is based on optical methods.
